(19) United States
(12) Patent Application Publication
(10) Pub. No.: US 2014/0330158 A1
(43) Pub. Date: Nov. 6, 2014

(54) BIOELECTRICAL IMPEDANCE MEASUREMENT METHOD

(71) Applicant: Tatung Company, Taipei (TW)

(72) Inventors: Shih-Jung Chang, Taipei (TW); Chao-Fa Lee, Taipei (TW)

(21) Appl. No.: 13/932,018

(22) Filed: Jul. 1, 2013

(30) Foreign Application Priority Data
May 6, 2013 (TW) 102116119

(51) Int. Cl. A61B 5/053; A61B 5/00

(52) U.S. Cl. CPC A61B 5/0531; A61B 5/0532; A61B 5/4872
USPC 600/548; 600/547

(57) ABSTRACT

A bioelectrical impedance measurement method is provided. A power signal and a frequency regulation signal for driving a measuring unit to perform bioelectrical impedance measurement are provided from a first sound channel output terminal and a second sound channel output terminal according to a measuring mode of a host device, and a signal measured and mixed is received by a microphone input terminal of the host device, and a bioelectrical impedance measurement result is analyzed and displayed by the host device.

### CROSS-REFERENCE TO RELATED APPLICATION

This application claims the priority benefit of Taiwan application serial no. 102116119, filed on May 6, 2013. The entirety of the above-mentioned patent application is hereby incorporated by reference herein and made a part of this specification.

### BACKGROUND

1. Technical Field

The invention relates to a measurement method. Particularly, the invention relates to a bioelectrical impedance measurement method.

2. Related Art

Today's measurement analysis of bioelectrical impedance is widely applied in different tests, for example, body fat test, human acupoint test, blood test, and enzymatic reaction of biotech, etc., and according to different test projects, operation frequencies in test analysis thereof are different, and the measurement analysis of bioelectrical impedance has advantages of simple, rapid, accurate, non-invasive, low cost, etc. The developed measurement apparatuses are only adapted to a single purpose, and do not have a composite mechanism capable of switching different test modes based on different measurement purposes, and can achieve a more accurate effect according to a multi-frequency measuring mode.

### SUMMARY

The invention is directed to a bioelectrical impedance measurement method, which is capable of implementing a plurality of bioelectrical impedance measurements, and is capable of being used in collaboration with a portable electronic apparatus such as a mobile phone, a tablet PC or a notebook computer, and has characteristics of lightness, slimness, shortness and smallness and is easy to use.

The invention provides a bioelectrical impedance measurement method including following steps. An alternating current (AC) power signal and a frequency regulation signal are respectively output through a first sound channel output terminal and a second sound channel output terminal of a host device according to a measuring mode set by the host device. A measuring unit is driven according to the AC power signal and the frequency regulation signal to perform bioelectrical impedance measurement on an object to be tested. A measurement result of the object to be tested is received through a microphone input terminal of the host device. The host device is used to analyze the measurement result to obtain biomedical information of the object to be tested.

In an embodiment of the invention, the step of driving the measuring unit according to the AC power signal and the frequency regulation signal to perform bioelectrical impedance measurement on the object to be tested includes following steps. The AC power signal is regulated to generate a direct current (DC) power signal. The measuring unit is driven according to the frequency modulation signal and the DC power signal to measure the object to be tested to generate a measurement signal.

In an embodiment of the invention, the step of receiving the measurement result of the object to be tested through the microphone input terminal of the host device includes mixing the AC power signal and the measurement signal to produce a mixed signal and outputting the same to the microphone input terminal of the host device.

In an embodiment of the invention, the bioelectrical impedance measurement method further includes displaying the biomedical information.

In an embodiment of the invention, the host device includes a mobile phone, a tablet PC or a notebook computer.

In an embodiment of the invention, the measuring mode includes an acupoint detection mode, a body fat measuring mode or a bio-detection mode.

According to the above descriptions, the power signal and the frequency regulation signal required for driving the measuring unit to perform measurement are provided through the first sound channel output terminal and the second sound channel output terminal of the host device, and the microphone input terminal of the host device is used to receive the measurement result, such that the host device can analyze the measurement result to obtain the biomedical information. In this way, the bioelectrical impedance measurement apparatus is adapted to multiple test projects, and can be used in collaboration with a portable electronic apparatus such as a mobile phone, a tablet PC or a notebook computer, and has characteristics of lightness, slimness, shortness and smallness and is easy to use.

In order to make the aforementioned and other features and advantages of the invention comprehensible, several exemplary embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a schematic diagram of a bioelectrical impedance measurement apparatus according to an embodiment of the invention.

FIG. 2 is a schematic diagram of a bioelectrical impedance measurement apparatus according to another embodiment of the invention.

FIG. 3 is a flowchart illustrating a bioelectrical impedance measurement method according to an embodiment of the invention.

### DETAILED DESCRIPTION OF DISCLOSED EMBODIMENTS

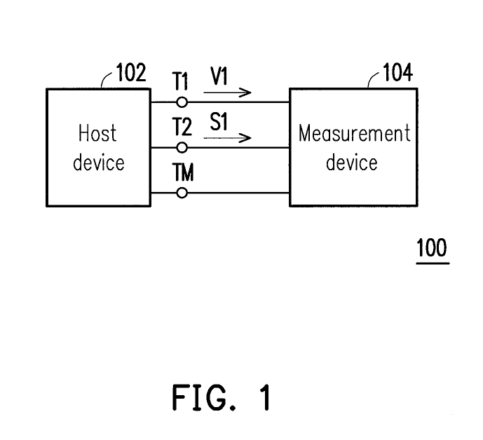

FIG. 1 is a schematic diagram of a bioelectrical impedance measurement apparatus according to an embodiment of the invention. Referring to FIG. 1, the bioelectrical impedance measurement apparatus 100 includes a host device 102 and a measurement device 104, where the host device 102 is, for example, portable electronic device such as a mobile phone, a tablet PC, or a notebook computer, etc., which has a sound channel output terminal T1, a sound channel output terminal T2 and a microphone input terminal TM. The host device 102 is coupled to the measurement device 104, which respectively outputs an alternating current (AC) power signal V1 and a frequency regulation signal S1 to the measurement device 104 through the sound channel output terminal T1 and the sound channel output terminal T2 according to a measuring mode of the host device 102, so as to drive the measurement device 104 to perform bioelectrical impedance measurement (for example, human acupoint detection, body fat measurement, skin humidity detection, etc.) on an object to be tested (for example, different parts of the body), where the measuring mode may include an acupoint detection mode, a body fat measuring mode, a bio-detection mode, etc. Moreover, the host device 102 further receives a measurement result of the object to be tested from the measurement device 104 through the microphone input terminal TM, and analyzes the measurement result to obtain biomedical information, for example, an acupoint skin impedance value and a body fat rate, etc. of the object to be tested.

In this way, the power signal and the frequency regulation signal are provided through the sound channel output terminal T1 and the sound channel output terminal T2 of the host device 102, and the measurement result is received by the microphone input terminal TM of the host device 102, such that the host device 102 can analyse the measurement result to obtain the biomedical information. In this way, the bioelectrical impedance measurement apparatus 100 is adapted to a plurality of test projects of different frequencies, and can be used in collaboration with a portable electronic apparatus such as a mobile phone, a tablet PC or a notebook computer, and has characteristics of lightness, slimness, shortness and smallness and is easy to use.

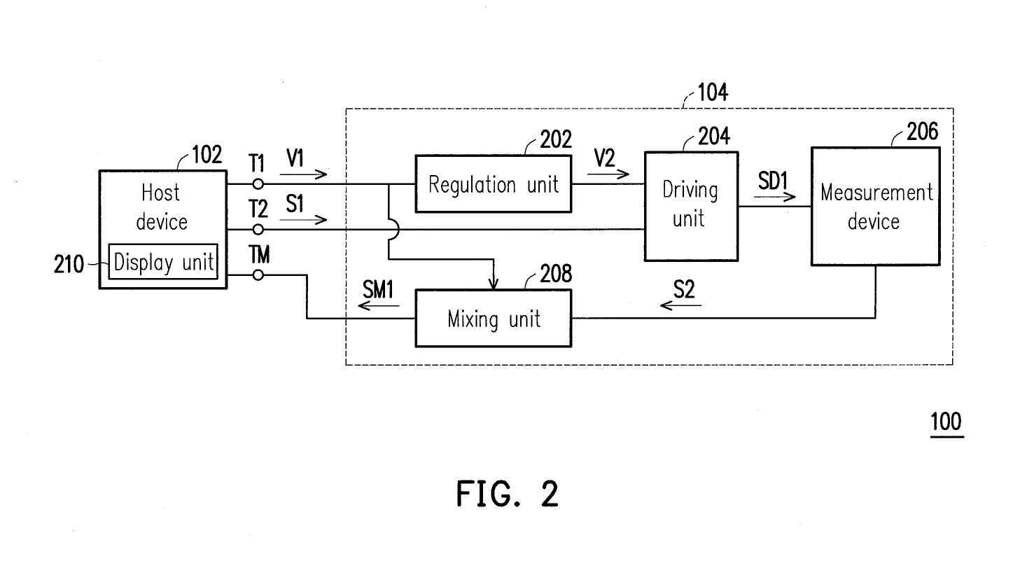

FIG. 2 is a schematic diagram of a bioelectrical impedance measurement apparatus according to another embodiment of the invention. Referring to FIG. 2, the measurement device 104 may include a regulation unit 202, a driving unit 204, a measuring unit 206 and a mixing unit 208. The regulation unit 202 is coupled to the sound channel output terminal T1 and the driving unit 204, the driving unit 204 is coupled to the sound channel output terminal T2 and the measuring unit 206, and the mixing unit 208 is coupled to the sound channel output terminal T1, the microphone input terminal TM and the measuring unit 206.

The regulation unit 202 receives the AC power signal V1 from the sound channel output terminal T1, and regulates the AC power signal V1 to provide a direct current (DC) power signal V2 to the driving unit 204. The driving unit 204 receives the DC power signal V2 from the regulation unit 202 and the frequency regulation signal S1 from the host device 102, and outputs a driving signal SD1 to the measuring unit 206 according to the DC power signal V2 and the frequency regulation signal S1, so as to drive the measuring unit 206 to measure the object to be tested. The measuring unit 206 outputs a measurement signal S2 obtained after measuring the object to be tested to the mixing unit 208. The mixing unit 208 mixes the received measurement signal S2 and the AC power signal V1 to convert the measurement signal S2 from a DC signal to an AC signal to produce a mixed signal SM1, and outputs the mixed signal SM1 to the microphone input terminal TM of the host device 102. The host device 102 can analyze the mixed signal SM1 to obtain the biomedical information of the object to be tested. Moreover, the host device may include a display unit 210, and the display unit 210 can display the biomedical information obtained by the host device 102 after analysing the mixed signal SM1.

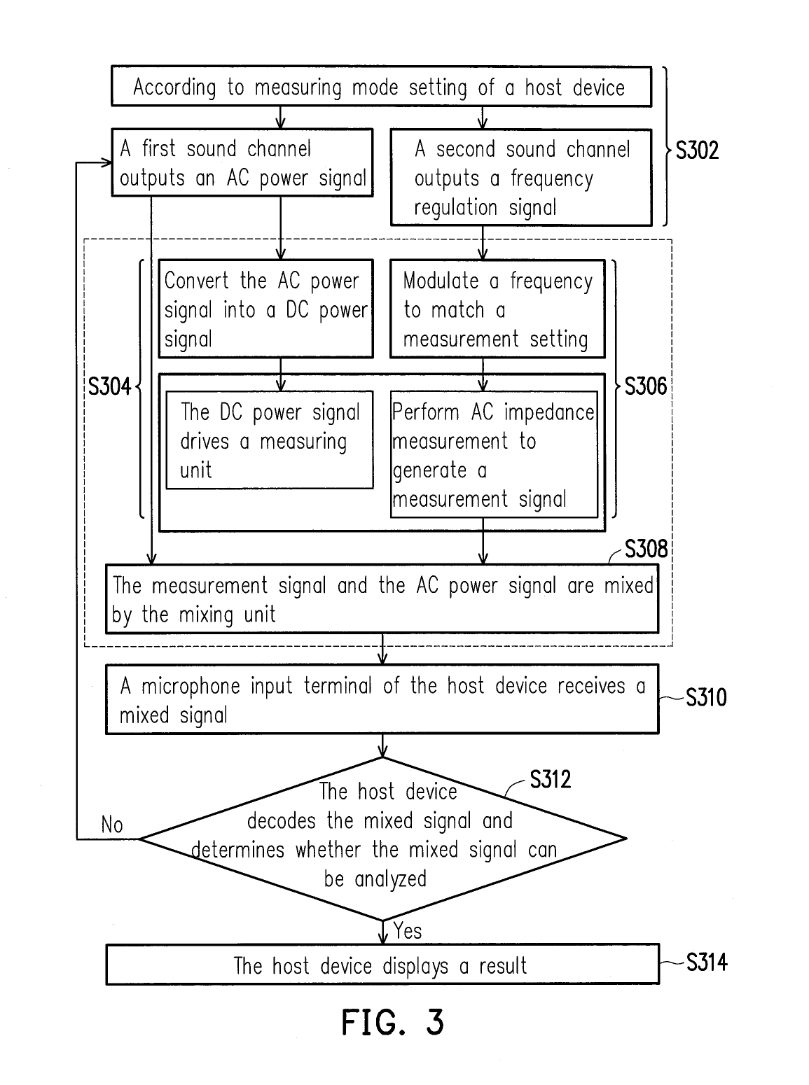

FIG. 3 is a flowchart illustrating a bioelectrical impedance measurement method according to an embodiment of the invention. Referring to FIG. 3, the bioelectrical impedance measurement method of the bioelectrical impedance measurement apparatus includes following steps. First, an AC power signal and a frequency regulation signal are respectively outputted through a first sound channel output terminal and a second sound channel output terminal of a host device according to a measuring mode of the host device (S302), where the first sound channel output terminal outputs the AC power signal to a regulation unit for converting into a DC power signal for driving a measuring unit (step S304), and the second sound channel output terminal outputs the frequency regulation signal set according to the measuring mode to the measuring unit, and the measuring unit performs the AC impedance measurement (to measure a bioelectrical impedance) to generate a measurement signal (step S306). Then, a mixing unit mixes the AC power signal output by the first sound channel output terminal with the measuring signal to generate a mixed signal (S308), and outputs the mixed signal to the microphone input terminal of the host device (step S310). Then, the host device decodes the mixed signal and determines whether the mixed signal can be analyzed (step S312), and if yes, the host device displays a result (step S314), and if not, a magnitude of the AC power signal is dynamically adjusted according to a magnitude of the mixed signal, for example, when the mixed signal is too large, in order to avoid distortion of an analysis result, the magnitude of the AC power signal is decreased, or when the mixed signal is too small to perform analysis, the magnitude of the AC power signal is increased. After the AC power signal is adjusted, the AC power signal is again converted into the DC power signal, and the measuring unit is driven and the measuring signal is mixed, and then the input terminal of the host receives the mixed signal, and the host decodes and determines the mixed signal to display a result.

In summary, the power signal and the frequency regulation signal required for driving the measuring unit to perform measurement are provided through the sound channel output terminals of the host device, and the microphone input terminal of the host device is used to receive the measurement result, such that the host device can analyse the measurement result to obtain the biomedical information. In this way, the bioelectrical impedance measurement apparatus is adapted to a plurality of test projects of different frequencies, and can be used in collaboration with a portable electronic apparatus such as a mobile phone, a tablet PC or a notebook computer, and has characteristics of lightness, slimness, shortness and smallness and is easy to use.

It will be apparent to those skilled in the art that various modifications and variations can be made to the structure of the invention without departing from the scope or spirit of the invention. In view of the foregoing, it is intended that the invention cover modifications and variations of this invention provided they fall within the scope of the following claims and their equivalents.

What is claimed is:

1. A bioelectrical impedance measurement method, comprising:
respectively outputting an alternating current power signal and a frequency regulation signal through a first sound channel output terminal and a second sound channel output terminal of a host device according to a measuring mode of the host device;
driving a measuring unit according to the alternating current power signal and the frequency regulation signal to perform bioelectrical impedance measurement on an object to be tested;
receiving a measurement result of the object to be tested through a microphone input terminal of the host device; and
using the host device to analyze the measurement result to obtain biomedical information of the object to be tested.

2. The bioelectrical impedance measurement method as claimed in claim 1, wherein the step of driving the measuring unit according to the alternating current power signal and the frequency regulation signal to perform bioelectrical impedance measurement on the object to be tested comprises:
regulating the alternating current power signal to generate a direct current power signal; and
driving the measuring unit according to the frequency regulation signal and the direct current power signal to measure the object to be tested to generate a measurement signal.

3. The bioelectrical impedance measurement method as claimed in claim 2, wherein the step of receiving the measurement result of the object to be tested through the microphone input terminal of the host device comprises:
mixing the alternating current power signal and the measurement signal to produce a mixed signal for outputting to the microphone input terminal of the host device.

4. The bioelectrical impedance measurement method as claimed in claim 1, further comprising:
displaying the biomedical information.

5. The bioelectrical impedance measurement method as claimed in claim 1, wherein the host device comprises a mobile phone, a tablet PC or a notebook computer.

6. The bioelectrical impedance measurement method as claimed in claim 1, wherein the measuring mode comprises an acupoint detection mode, a body fat measuring mode or a bio-detection mode.
